(19) United States
(12) Patent Application Publication
(10) Pub. No.: US 2008/0014291 A1
(43) Pub. Date: Jan. 17, 2008

(54) Process for the Production of Multi-Component Mineral Substance Preparations

(75) Inventor: Andreas Noack, Mainz (DE)

Correspondence Address: DEMONT & BREYER, LLC, 100 COMMONS WAY, Ste. 250, HOLMDEL, NJ, 07733, US

(73) Assignee: Noack Enterprises, Mainz Laubenheim (DE)

(21) Appl. No.: 10/595,993

(22) PCT Filed: Nov. 25, 2004
(86) PCT No.: PCT/EP04/13391

§ 371 Date: Jun. 7, 2007

(30) Foreign Application Priority Data
Nov. 25, 2003 (DE) 10355400.9

(51) Int. Cl. A61K 36/00; A23L 1/304; A61P 37/00; A61P 43/00; A61P 9/00; C12N 5/00

(52) U.S. Cl. 424/725; 435/410

(57) ABSTRACT

Process for producing mineral substance preparations with multiple components that contain a low level of or no contaminants wherein material of plant or animal origin is used and at least two different thermo-oxidative treatment steps in separate locations are carried out.

Mineral substance preparations produced according to the invention are characterized in that they basically contain the mineral substances that the respective plant absorbs from the ground in a highly bio-available form, at the same time have a very low content of thermally-modified organic compounds.

Not easily dissolvable a priori trace elements are therefore preferably in colloidal form, which is why biologic absorption is especially high. The effect of these mineral substance preparations on health and fitness are by far superior than the currently available mineral substance preparations, since they also all contain trace elements that can no longer be analytically verified but can nevertheless have a considerably energizing effect on the human body.

The submitted invention concerns a process for the production of multi-component mineral substance preparations.

The production of plant minerals for health reasons is known about and understood, particularly the use of incineration processes. Plant minerals have the advantage over synthetically composed mineral substance preparations of containing a broader and more homogeneous mineral substance spectrum, whereby based on the fundamental concept it is guaranteed that the danger of an overdose of individual minerals hardly exists, as long as each nutritional physiological dose represents a sensible amount of the plant in question.

In accordance with DE 3727417A, mineral substance preparations are simply created by the incineration of organic or plant materials. Thereafter the preparation in question is simply produced by a discontinuous temperature treatment of plant materials up to 920° C.

However, the problem with this lies, above all, in the area of the process technique, mainly in the efforts to obtain an extremely pure preparation free of harmful substances. In a technically sound executable process it is essential to decontaminate the product discontinuously through by-products which can have here a powerfully toxic effect. In addition, it is very desirable from the nutritional and physiological point of view that the mineral substance matrix be virtually completely insoluble, to transfer over to bio-available forms. This theme will however not be examined here.

In JP 04016164A, the embodiment of mineral substance preparations based on incinerated organic substances is described, whereby the ashes extracted are either acidulous or neutral. Here too, neither the problem of harmful substances produced in the incineration process, nor the possibilities concerning how the non-extractable mineral substance component is obtained is examined.

According to JP 2001292725A, in order to obtain a neutral salt with high solubility, one extracts a plant with the name salicornia using hot water, then dries and incinerates the extract for 4 hours. Using this method easily soluble minerals like potassium and sodium are obtained. However, on the other hand and for the most part, rare element trace minerals are not released by water extraction from raw plant material and therefore also do not end up in the product, by which process on the other hand the natural, plant-filtered mineral spectrum becomes distorted.

In summary, it may be said concerning the current method or prior art is dependent on the quality, i.e. the usability or the toxicity, of mineral substance preparations based on ash which are very sensitive to contamination by harmful substances, which is neither discussed nor addressed in the processing technique.

In addition, it can be determined that the process described in the literature is exclusively carried out in batch processes and this with very small product quantities. The developed processes do not allow any technical up-scaling.

The technical solution involving the incineration of substances free of harmful elements is absolutely and critically dependent upon the pure product not being contaminated with half-complete material; the complexity is due to the fact that the intermediate product, the primarily oxidized material, is highly toxic and any contamination of the end product must be herewith excluded, i.e. the transport of the mineralized product in the process must be completely controllable and reproducible.

The immense volume reduction during the incineration process is especially difficult, where an inhomogeneous mixture of completely incinerated and only partially incinerated areas results. Therefore, the situation whereby evaporating and harmful gasses are absorbed into the ashes and thereby contaminating them is a problem which is it is not possible to overcome with the help of the present state of the equipment.

The purpose of the submitted invention is the creation of a multi-component mineral substance preparation, which is as complete as possible, and which also contains all minerals which guarantee and strengthen the vitality of our bodies and which is free of substances which may be a health concern, as well as the details of the related process. The fundamental factor is that the product is already guaranteed free of harmful substances during the production process, since for example a crystallization process cannot be applied as a purification step because important trace elements/minerals would be lost in the process.

These problems are solved in accordance with the invention by the distinctive features of the Independent Patent Claims.

The process according to the invention for the production of multi-component mineral substance preparations, which are free of harmful substances, contains in particular the following steps:
a. Primary thermal-oxidative treatment of organic raw materials, of plant or animal origin, in a primary temperature treatment zone;
b. Transport of the product of the primary thermal-oxidative treatment into a second temperature treatment zone;
c. Secondary thermal-oxidative treatment of the product of the primary thermal-oxidative treatment in a second temperature zone.

On the grounds of simplicity, the following will be abbreviated thus:
Primary thermal-oxidative treatment will also be referred to as primary oxidation. Secondary thermal-oxidative treatment as secondary oxidation.
The product of the primary thermal-oxidative treatment as primary oxidate.
The product of the secondary thermal-oxidative treatment as secondary oxidate.

The primary thermal oxidative treatment involves the usual incineration, whereby the focus in terms of the technical process is on how the ashes, in other words, the primary oxidate, can be made as powdery as possible and therefore the oven parameters can be set in such a way that when implementing autothermic operation as extensively as possible, a safe margin in relation to the ash melting temperature is maintained, so that furnace ovens can be used.

The secondary thermal oxidative treatment, however, has the aim of reducing or minimizing the part of the remaining organic compounds and of carbonized carbon below the level that would give cause to health concerns using an autothermic method, as well as converting insoluble components of the primary oxidate into rudimentarily soluble forms.

The very insoluble residues themselves contain valuable trace and ultra-trace elements, which should of course be solubilized to the highest possible extraction. Here it has been shown that the process requirements, in which the baking or the melting of powder particles is achieved and by which these rudimentarily soluble forms occur to a heightened yield, are extremely useful.

The term “rudimentarily soluble forms” means those forms, which when soaking the secondary oxidate with acid, in particular with citric acid, become soluble or colloidially soluble. Therefore as a rule, the secondary thermal oxidative treatment is performed at a higher temperature than the primary thermal oxidative treatment, typically at least 10° C., preferentially 30° C. and especially preferred at least 50° C. above the temperature of the primary thermal oxidative treatment. Nevertheless in individual cases the temperatures can also be more than 200° C. above the temperature of the primary thermal oxidative treatment.

The tendentially resultant granulate and/or the molten material in the secondary thermal oxidative treatment is pulverized once again into a preferable embodiment of the invention.

Using the invention process, it is possible to raise the acid solubility to at least 95%, preferably to at least 98%, and most preferably to above 99%, which in turn has the effect that a priori forms which are difficult to dissolve are over-proportionally solubilized and bio-available.

According to EP 0 891718—example 1—one obtains in a typical incineration process, even with raw materials with a relatively high soluble ash component, at best a 90% solubility of the ash deposits in acid. Depending on the raw material, an incineration temperature is selected which produces the most powdery and workable form of primary oxidate. The selected temperature can be between 500° and 3000° C., preferably between 600° C. and 1200° C., and most preferably is between 750° C. and 1050° C.

The primary oxidant typically shows a maximum of 30%, preferably a maximum of 10% and most preferably a maximum of 3% of the original carbon content as organically combined carbon.

As a rule, the primary oxidate is cooled off and sieved, typically with a sieve size smaller than 1 mm, although preferably with a size smaller than 0.2 mm, optionally and temporarily stored and then processed in a second temperature treatment zone. The separation in situ of both temperature treatment zones can take place in an integrated production facility in the framework of a superordinate production plant which integrates both these zones, but the processes can also take place in two completely different production locations.

As organic raw materials, plant or animal origin, not only all seed plants (spermatophyte family) may be used but also algae. Also usable are seed plants, e.g. root, tuber, bulb, stem, leaf, seed and fruit types. Forage plants such as grasses and beans may also be used. Plants with medicinally beneficial properties, e.g. field horse-tail (Equisetum arvense), adonis (Adonis), mandrake (Mandragora), arum (Arum), lemon peel, olive husks, wood, nettle, spinach, sugar beet, etc. may also be used. See table 1

TABLE 1

Artichoke
Horehound
(Marrubium vulgare)
Angelica
Aniseed
Apple
Apfelkraut (Herba Marrubii),
see Camomile
Arnica
Brook mint
(mentha aquatica)
Valerian
Balsam fir
Bearberry
(Arctostaphylos uva-ursi)
Common
clubmoss (Lycopodium clavatum)
Ramsons
(Allium ursinum)
Spicknel (Meum athamanticum)
Basil
Comfrey
Burnet (Pimpinella saxifraga)
Henbane (Hyoscyamus niger),
Birch (Betula lenta)
and moor birch
(Betula pubescens)
Pear
Marsh clover
(Menyanthes trifoliata)
Bittersweet
(Solanum dulcamara LINNÉ)
Hemp (Cannabis sativa L.)
Spiny restharrow
(Ononis spinosa)
Houseleek (Sempervivum)
Hazelnut
Bilberry (Vaccinium myrtillus)
Skullcap
(Scutellaria lateriflora)
Meadow saffron
(Colchicum autumnale)
Motherwort (Leonurus cardiaca)
Hay flowers (Graminis flos)
Raspberry
Shepherd's purse
(Capsella bursa-pastoris)
Elder (Sambucus)
Elderflower
Hops (Humulus)
Sweet clover
(Melilotus officinalis)
Coltsfoot
(Tussil\acute{a}go f\acute{a}rfara)
Dog rose (Rosa canina)
Icelandic moss
(Cetraria islandica)
Coffea arabica (Coffea arabica)
California poppy
(Eschscholzia californica)
Common sweet flag
(Acorus calamus)
Camomile
(Herba Marrubii))
Rose
Rosemary
Horse-chestnuts (A\acute{e}sculus)
Saffron
Sage
Woodland germander
(Teucrium scorodonia)
Common sea-buckthorn
(Hippophae rhamnoides)
Strawflower (Helichrysum
arenarium (L.) Moench)
European barberry
(Berberis vulgaris)
Horsetail (Sphenophyta)
Yarrow (Achill\acute{e}a)
Hemlock (Apiaceae)
Opium poppy
Primrose
Echinacea
(Echinacea angustifolia)
Chive (Allium schoenoprasum L.)
Greater celandine
(Chelidonium majus)
Black elder (Sambucus nigra)
Black tea
Iris (Iris)
Celeriac (Apium graveolens)
Stemless carline thistle
(Carlina acaulis)
Blueberry (Vaccinium Cyanococcus)
Wolfsbane (Aconitum)
Purple loosestrife
(Lythrum salicaria)
Tormentil (Potentilla erecta)
Savory (Satureja)
Borage (Borago officinalis)
Nettle (Utica)
Blackberry
(Rubus fruticosa agg.)
Smooth rupturewort
(Herniaria glabra)
Christ's thorn
(Paliurus, spina-christi)
Christmas rose
(Helleborus niger)
Dill (Anethum graveolens)
Burning bush (Dictamnus albus)
Good King Henry
(Chenopodium bonus Henricus)
Oregano (Origanum vulgare)
Sweet Violet (Viola odorata)
Rowan (Sorbus aucuparia)
Southern wood
(Artemisia abrotanum)
English hawthorn
(Crataegus laevigata
‘Paul's Scarlet’)
Gypsy weed (Veronica)
Ivy
Yew
Marshmallow
(Althaea officinalis)
Wolfsbane (Aconitum)
Verbena
(Verbena officinalis)
Strawberry
Tarragon
Eucalyptus
Buckthorn
Fig
Fennel
Buckbean
(Menyanthes trifoliata)
Foxglove
Flax (Linum usitatissimum)
Lady's mantle
Common daisy (Bellis perennis)
Silver weed
(Potentilla anserina)
Poison arum
Yellow gentian
Yarrow or bloodwort
(Achill\acute{e}a millef\acute{o}lium)
Evening primrose
Ground-elder
(Aegopodium podagraria)
Ginseng
Goldenrod
Pomegranate tree
Great burnet
(Sanguisorba officinalis)
Ground ivy
(Glech\acute{o}ma heder\acute{a}cea L.)
Bugle plant (\acute{A}juga)
Good King Henry
(Chenopodium bonus-henricus)
Oats
Poplar
Catechu (Acacia Catechu)
Corn poppy (Papaver rhoeas)
Salad burnet
(Sanguisorba minor)
Burdock
Garlic
Mullein
Bluebottle
Pasqueflower
Pumpkin
Lavender
Lovage (Levisticum officinale)
Lime-tree blossom
Linden
Lime-tree blossom
Dandelion
Bay (Laurus nobilis)
Lungwort (Pulmonaria)
Lilly of the valley
(Convallaria majalis)
see violet
Mallow (Malvaceae)
Milk thistle
(Silybum marianum)
Sweet violet (Viola odorata)
Horse radish
Sea squill (Urginea maritima)
Masterwort (Peucedanum)
Mistletoe
Chaste tree
Evening primrose
Hellebore (Hell\acute{e}borus)
White hellebore
(Veratrum album)
Agrimony
Oleander
Olive leaves
Oregano
Birthwort
(Aristolochia clematitis)
Palm lily
Passion flower
Peppermint
Peony
Pimpernel
Portulaca
Purple coneflower
(Echinacea purpurea)
Wild thyme
(Thymus serpyllum)
Quinoa
Quince
Tansy
Rue
Rhubarb root
Soy bean
Coneflower
Capsicum
Asparagus fern
Liquorice
Ribwort
Jimson weed
Holly
Pansy
Tobacco
Centaury
Tea shrub
Thyme
Banewort
Tomato
Morning glory
Martagon lily
(Lilium martagon)
Violet
Rowan tree
Chickweed
Juniper
Scots pine
(Pinus sylvetris)
Wild sage
(Teucrium scorodonia)
Woodruff (Galium odoratum)
Clematis
Comfrey
Walnut tree
Gravel root
Chicory (Cichorium)
Willow bark
Whitethorn
White mustard seeds
White hellebore
(Veratrum album)
Silver fur
Wormwood
Basswood
Grecian foxglove
(Digitalis lanata)
Castor oil plant
(Ricinus communis)
Gall clover
Ferm
Hyssop
Red Virginia creeper
Horse tail
(Equisetum arvense)
see field horse tail
Hedge rose
Red currant
St. John's wort
Winter cherry
Castor oil plant
(Ricinus communis L.)
Mangold
Red foxglove

Less preferably, but entirely possible are special animals or animal parts such as bones or special organs. From a medicinal standpoint the use of special plants or parts of animals is therefore important since there are those who would specifically like to implement the “natural” enrichment of a number of trace elements for therapeutic purposes. Especially high quality raw materials include grasses from certain mountain pastures that have not hitherto been intensively cultivated as well as sea algae.

Other high quality raw materials include leaves such as olive tree leaves, maple leaves, birch leaves, stinging nettle, aloe vera, thyme, etc. Especially preferable are root and root parts, for example, of wild and/or forest herbs, ginseng, etc. Also fungi and mycelium. Advantageous is in particular the use of extracted or compacted remains, as these represent very high quality mineral substances.

The secondary thermal oxidative treatment is preferentially carried out in a pushed-batt oven, a roller furnace or a conveyor furnace quasi-continuously. Also a externally heated revolving tube or an externally heated tube with a spiral conveyor can be used continuously. Unlike the primary thermal oxidative treatment, the secondary thermal oxidative treatment can be controlled by external heat sources, or optionally also controlled using microwaves.

In accordance with the invention, the process may be carried out using batch operation with two different heating zones, however an at least partly continuous processing is preferable, whereby preferentially the secondary oxidation is carried out continuously.

According to the invention, the process additionally concerns a variant, by which at the site of the harvest the plants can already be primarily oxidized, the primary oxidate then transported to the site of the secondary oxidation and there be further processed according to the defined process.

The secondary thermal oxidative treatment can thereby be carried out in normal air, although it is advantageous to carry out the process with enriched or pure oxygen and/or water steam. The advantage of an enriched oxygen or water steam atmosphere is, on the one hand, the higher oxidative potential of the atmosphere, which supports the oxidation of the a priori inert precious metals, which thereby become better bio-available. In addition, the formation of nitric oxides is inhibited which otherwise would have a considerable toxic affect on the product.

Depending on the temperature range, the retention time in the second temperature treatment zone is typically set from 20 minutes up to 100 h, preferentially 2 h to 36 h. The simplest form of grinding can be carried out in a mortar or grinding mill. The grinding process can thereby be carried out in a dry state, however, it is advantageous to employ a wet process by which, as a rule, the particle size may be reduced much further. A dry and wet grinding process may also be combined. The wet grinding process is achieved preferably in a pH range of between 8 and 16, and especially preferably between 10 and 14. In this pH range, the ground substance has the lowest tendency to agglomerate. The grinding energy to be used in the grinding process is between 0.1 and 10 Wh/kg when using a bead mill.

The ground substance can further have organic acids added, in particular citric acid, vinegar, malic acid, ascorbic acid, lactic acid, acetic acid and vinegar, as well as similar mixtures.

Using a dry grinding process, it can be advantageous to fill the ground substance powder into capsules/gelatine capsules or compressed into tablets with a binding agent, preferably lactose.

By adding organic acids to the ground substance, in particular citric acid and/or malic acid and/or lactic acid and/or acetic acid, and also similar mixtures, colloidal solutions may be produced containing many trace elements and ultra-trace elements in cluster size which are for the most part smaller than 100 nm, as shown in illustration 1.

According to the invention, the mineral substance preparations may be used as nutritional supplements, food additives, beauty aids, medication, hereby the intake can be in particular either in oral, inhalative, intravenous, rectal or topical form.

Also advantageous from a health point of view is the case, whereby a juice or an extract made from the original raw material, or optional as a dried powder, finally a mineral solution created in accordance with the invention preferably in the described acidified form, combined with a foodstuff or a nutritional supplement, a balanced diet or an additive, which contains the most important nutritional physiological substances of the plant in question in highly concentrated form.

Mineral substance preparations produced accordingly can of course be mixed with other minerals, wherein one would normally mix at least 2% of the mineral substance preparation produced in accordance with the invention in with the usual commercially-available mineral substance preparations, whereby the balance of the commercially-available preparations is clearly increased.

Of course, also the quality of any food can be improved by adding mineral substance preparations produced according to the invention.

Mineral substance preparations that are produced according to the process described in the invention are suitable as a broad spectrum medication also for remedies for which colloidal silver is recommended. Preparations produced according to the invention are especially effective for the following indications:
Heavy metal poisoning, chronic nausea, migraine, allergies, cardio-vascular diseases, high blood pressure. In connection with this, experts have observed a strong correlation between a deficiency in trace elements and the occurrence of disease.

The advantages of the process with respect to prior art can be described in summary as follows: Firstly multi-component mineral preparations of natural origin, which are free of harmful substances, are technically producible and available. A balanced, natural graduation of minerals and trace elements, as an optimal nutritional supplement to frequently demineralized food, helps refill the body's own mineral substance stores. In this way, users strengthen their immune system. A “natural mineral substance cocktail” is better tolerated during mineralization cures, without health-damaging situations occurring, since no unilateral strain can develop. Due to the high potassium/sodium levels body fluids are alkalized, i.e. the body pH increases and waste products are released resulting in the following:
Good blood pressure regulation; a prophylactic effect against cardio-vascular diseases; an increase in cell oxygenation; an increase of the energy in the body with an increase in the level of health and fitness.

### APPLICATION EXAMPLE

A mixture of wood was treated in a normal furnace oven and incinerated, i.e. treated in a primary thermal oxidative process at a temperature of approx. 800° C. The primary oxidate was sieved to 0.1 mm and still contained visible amounts of carbon particles which in particular, when the primary oxidate was put into water, collected on the water's surface. The sieved primary oxidate was placed in a porcelain crucible and then treated in a muffle kiln at 1100° for 24 h using a thermal oxidative process with oxygen flushing. The secondary oxidate was essentially molten and then scraped out of the crucible after cooling off. The secondary oxidate was ground in a ball mill and sieved to 0.1 mm. It contained neither visibly perceptible nor detectable organic or carbonized substance impurities when thermally analyzed.

The powder thus obtained is suitable for mixing into a wide variety of foodstuffs, for compressing into tablet form using, e.g. lactose or for filling gelatin capsules, etc. Additionally 1 kg of the secondary oxidate was pre-ground with 10 l of water in a grinding bead mill with beads of between 1.5 to 2.5 mm in size using an energy value of 0.5 Wh/kg (solid substance) and in a second pulverizing process was ground with beads of 0.3-0.4 mm in size using an energy value of 3 Wh/kg (solid substance).

2.0 g of the suspension obtained in this manner were set using citric acid to a pH value of between 3 and 4 and diluted with 150 ml water. The colloidal solution thus obtained was characterized by means of diffraction measurements (see Illustration 1) and examined using inductively coupled plasma mass spectroscopy (see Table 2).

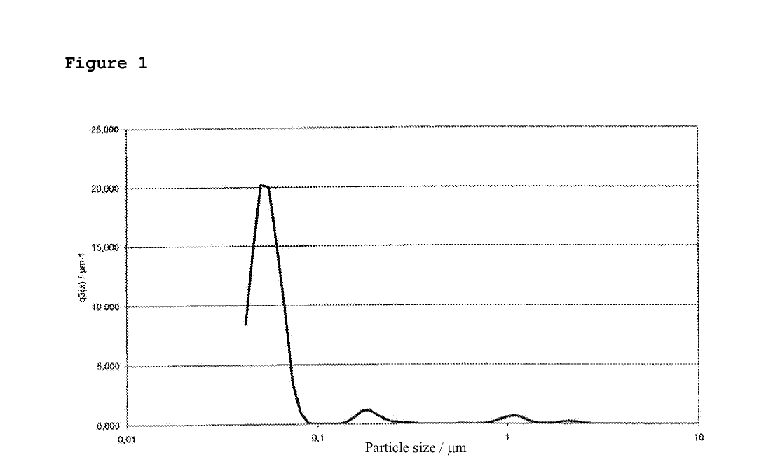

FIG. 1 note: the measurements were made by means of a laser diffraction spectroscope according to the PIDS method (Polarization Intensity Differential scattering) with a spectrometer of the type LS230 made by the firm Beckmann-Coulter GmbH/Krefeld, where the particle spectrum was calculated and recorded in relation to the volume density distribution q3 (see: H. Rumpf, “Mechanical Process Technique”, Carl Hanser Verlag/Munich/Vienna, 1995, p. 12-15).

TABLE 2

| | |
|---|---|
| Element | Wood (μg/g) |

| | | |
|---|---|---|
| Ti | Titanium | 1560.10 |
| V | Vanadium | 35.56 |
| Cr | Chrome | 6462.90 |
| Mn | Manganese | 12775.48 |
| Fe | Iron | 24672.32 |
| Co | Cobalt | 19.87 |
| Ni | Nickel | 1062.50 |
| Cu | Copper | 458.90 |
| Zn | Zinc | 459.88 |
| Ga | Gallium | 9.88 |
| Ge | Germanium | 3.51 |
| As | Arsenic | 1.01 |
| Y | Yttrium | 288.40 |
| Zr | Zirconium | 5060.75 |
| Nb | Niobium | 5.62 |
| Mo | Molybdenum | 43.13 |
| Ru | Ruthenium | 0.00 |
| Pd | Palladium | 73.10 |
| Ag | Silver | 29.26 |
| Cd | Cadmium | 23.05 |
| In | Indium | 0.00 |
| Sb | Antimony | 2.87 |
| Te | Tellurium | 7.63 |
| La | Lanthanum | 14.42 |
| Ce | Cerium | 31.64 |
| Pr | Praseodymium | 3.69 |
| Nd | Neodymium | 11.44 |
| Sm | Samarium | 2.43 |
| Eu | Europium | 0.67 |
| Gd | Gadolinium | 1.73 |
| Tb | Terbium | 0.32 |
| Dy | Dysprosium | 1.74 |
| Ho | Holmium | 0.31 |
| Er | Erbium | 0.83 |
| Tm | Thulium | 0.13 |
| Yb | Ytterbium | 1.60 |
| Lu | Lutetium | 0.00 |
| Hf | Hafnium | 127.85 |
| Ta | Tantalum | 0.52 |
| W | Tungsten | 13.68 |
| Re | Rhenium | 0.00 |
| Os | Osmium | 0.00 |
| Ir | Iridium | 0.00 |
| Pt | Platinum | 1.20 |
| Au | Gold | 0.00 |
| Hg | Mercury | 0.00 |
| Tl | Thallium | 0.00 |
| Pb | Lead | 13.06 |
| Bi | Bismuth | 0.47 |
| Th | Thorium | 7.04 |
| U | Uranium | 3.21 |
| B | Boron | 1351.22 |
| Al | Aluminum | 38443.71 |
| Si | Silicon | 53238.96 |
| P | Phosphorus | 2713.10 |
| S | Sulfur | 14701.15 |
| Se | Selenium | 0.00 |
| Br | Bromine | 0.00 |
| Li | Lithium | 52.20 |
| Be | Beryllium | 0.00 |
| Na | Sodium | 19883.98 |
| Mg | Magnesium | 60715.70 |
| K | Potassium | 106809.43 |
| Ca | Calcium | 252354.22 |
| Sc | Scandium | 4.02 |
| Rb | Rubidium | 119.29 |
| Sr | Strontium | 2017.13 |
| Cs | Cesium | 1.31 |
| Ba | Barium | 1678.80 |

Further aspects of the invention are presented in the following text.

According to the current state of the art, mineral preparations are mainly mixed together from individual salts. The basis of the composition of the mixture is the assumption that ingesting a determined dose of minerals substantially present in the body is sensible; however the composition of the mixture is consequently rather arbitrary, since medical expertise is not advanced enough to determine in particular what dosage recommendations for trace elements are generally valid. Trace elements such as zinc, molybdenum, chrome, gold, rhodium, platinum, nickel, cobalt, iridium, osmium are as a rule not taken into account in mineral substance preparations with the result that a “synthetic” mixture of salts of various origins means that trace elements always administered in unnatural doses, especially also in relation to the “primary minerals” such as calcium, magnesium, and potassium.

There are indeed many opinions concerning the optimum dosage of minerals, however combining them is a science in itself; the necessary knowledge for this fills whole volumes of specialized literature. In particular, the influence of trace elements on our health and our well being is very difficult to determine; the possibilities of absolute and relative dosage of the trace elements in relation to each other are completely limitless. The big problem with dose recommendations for trace minerals is the insufficient analysis in the ultra-trace area, where one can basically only state that specific ions such as iridium, rhodium, platinum, gold, etc. occur in natural foodstuffs below the detectable limit—but only based on the chemistry of these forms. When considering the catalytic properties of their homogeneously dissolved complex forms, it is known that these forms are not comparable to anything else.

Even the most minute traces of specific interim metal complexes can have dramatic results and trigger the occurrence or non-occurrence of specific internal body reactions.

Since in all probability after an erroneous overdose of trace elements, even in extremely small amounts, serious consequences will occur, and these forms will then be removed from the range of commercially-available mineral substance preparations. The consequence is that such mineral substance preparations are relatively lacking in trace minerals.

Especially critical here is that due to intensive agriculture in industrial countries, the trace element content of fields is already very low anyway which also means that the trace element content of vegetables and fruit has been massively weakened. Experts also estimate that our own body mineral deposits reduce significantly with age, which is a main factor for the increase in immunodeficiency diseases and cancer. Experts assume that synthetically composed mineral products, which often contain high doses of calcium, give cause for concern on health grounds: mineral substances have in particular the job of keeping body pH values as high as possible, to counteract chronic acidity, which more than 80% of the population in industrial countries suffer directly or indirectly and which represent the cause of cardiovascular diseases, fungal diseases, parasite infestation and for reduced immune system activity. Acidosis is the increase of acid in the body's fluids, mainly of the lymph, as a result of nutrition; foods from which mineral substances have been removed such as flour and sugar are responsible for high acid levels.

Over the years, these acids in connection with alkaline earth deposits collect in particular calcium, as solid slag, also called poly-molecular alkaline earth salts, in our body and prevent the free circulation of blood and therefore the transport of oxygen into our cells. If one wishes to reverse this slag deposit process or least to slow it down, one has to use mineral substance preparations in order to raise the body's pH value and dissipate the poly-molecular alkaline earth salts which are difficult to dissolve.

This is exactly the effect that the simply mixed mineral salt preparations rich in calcium and magnesium do not have. Alkaline earth salts which are difficult to dissolve are not dissolved by even more alkaline earth. The opposite is the case: if one uses mineral substance preparations containing high levels of calcium and magnesium in order to raise the body's pH value, and then uses even higher doses, an acute and very dangerous build up of further slag salts, which can greatly hinder or block the cardiovascular system, can occur.

A further problem with the application and dosage of minerals is posed by those forms, which interfere with each other and should not be taken together. For example, molybdenum is considered an antagonist for cobalt, while copper on the other hand is considered to be an antagonist for zinc; see A Beginners Introduction to Trace Minerals, Erwin Dicyan Keats Publishing, Inc., New Canaan, Conn./USA.

When one considers that there currently more than 87 known trace elements, whose dosage can be altered, it becomes clear that the possibilities for synthetically mixing mineral substance preparations are infinitely great.

Therefore, if one only wanted to test the biological value in the most prevalent synthetic mineral substance preparations in long term test, the length of time required would hardly be quantifiable. Then there is the problem of dosing trace elements: the bio-availability and effectiveness in various modifications. Therefore the trace elements stored in edible plants are present, for example, in colloidal form, i.e. in metal cluster form. According to the opinion of leading experts, these clusters are much more valuable for our organisms than the corresponding salts. The best advice for composing mineral substance preparations is therefore to use of the actual mineralization of plants.

This brings us to the second approach for producing mineral substance preparations, the production of what are known as ash products. Here an attempt is made to incinerate mainly raw plant materials to obtain a balanced, naturally filtered mineral substance preparation.

In accordance with DE 3727417A, mineral substance preparations are created simply by the incineration of organic or plant materials. Thereafter the corresponding preparation is simply created by a discontinuous temperature treatment of plant materials up to 920° C.

The problem with this however lies above all in the area of the process technique, mainly in the efforts to obtain an extremely pure preparation free of harmful substances. On the one hand, one is tempted to achieve a decomposition of the organic materials which is as complete as possible by using the highest temperature possible; however, on the other hand, one wants the temperature to be kept as low as possible in order to prevent the formation of nitric oxides. Using state of the art discontinuous and one-step incineration methods, extreme temperature fluctuations occur within the incinerated sample. This leads either to absorption of harmful substances in cold spots in the raw materials or to an incomplete oxidation of the raw materials; in the long run, both effects can render the entire product unusable. A suitable process technology therefore has a decisive influence on whether the product promotes health or whether it has a toxic effect. In no way does the indicated state of the art take into account the contamination by nitrate/nitrite or organic residual components.

A further problem with the current state of the art is the bio-availability of the trace minerals, which according to expert opinion is especially great if these trace minerals are present in a metallic state as a colloid. Some experts suspect that a cluster size of between 10 and 100 atoms represent the optimum for our bodies. In the current state of the art, this is not explored.

In JP 04016164A, the preparation of mineral substance preparations based on incinerated organic substances is described, whereby the ashes are extracted either acid based or neutrally extracted. However, the problems of harmful substances arising from the incineration process are not addressed.

In order to obtain a neutral salt with high solubility, one extracts a plant with the name salicornia in accordance with JP 2001292725A using hot water, then dries and incinerates the extract over the course of more than 4 hours. This process obtains above all the easily soluble minerals such as potassium and sodium, whereas the trace minerals of the rarer elements are not released by the water extraction from the raw material of the plant and therefore do not end up in the product, whereby the natural, plant filtered mineral spectrum becomes distorted.

In summary, it may be said concerning the current state of the art that the quality, i.e. the usability or the toxicity, of mineral substance preparations based on ash is very sensitive to and dependent on contamination by harmful substances. In addition, it is to be determined that the process described in the literature is exclusively carried out in batch processes and this with very small product quantities.

This process does not allow any technical up-scaling, since the limited thermal conductivity and the dynamics of the flow and diffusion of a probe in the gram range are completely different from those in the kg range or in the ton range. The technical solution involving the incineration of substances free of harmful elements is absolutely critically dependent upon the pure product not being contaminated with half complete material; the complexity is in the fact that the intermediate product, the primarily oxidized material, is highly toxic and any contamination of the end product must herewith be excluded, i.e. the transport of the mineralized product in the process must be completely controllable and reproducible. The immense volume reduction during incineration process is especially difficult, where an inhomogeneous mixture of completely incinerated and only partially incinerated areas results. Also the fact that evaporating harmful gases are absorbed by the ashes and thereby contaminate them is a problem which is not controllable using the state of the art.

The fundamental advantage is that the preparation according to the invention is already guaranteed free of harmful substances during the production process, since for example a crystallization process cannot be applied as a purification step because important trace elements/minerals would be lost in the process.

The process according to the invention for the production of multi-component mineral substance preparations, which are free of harmful substances, contains in particular the following steps:
a. Primary oxidation of organic raw materials of plant or animal origin in of a primary temperature treatment zone
b. Conditioning of the primary oxidized materials
c. Transport of the primary oxidized materials into a secondary temperature treatment zone
d. Secondary oxidation of the primary oxidized materials in a secondary temperature treatment zone

Furthermore the invention describes an apparatus for producing mineral substance preparations with the following features:
a. An apparatus for introducing primary oxidized material of plant or animal origin
b. A conveyor unit by which the primarily oxidized material is transported
c. A transport channel in which the primarily oxidized material is transported
d. A heating zone
e. An exhaust air channel

Due to the two-phase nature of the process, a defined retention time behaviour for all elements to be mineralised, especially in the secondary oxidation process, is achieved, which makes it easier to obtain an uncontaminated end product. The primary phase is carried out in a temperature range of between 500° and 3000° C., whereby the organic material is preferably oxidized with air at 500°-1500° C. In this process, a maximum of 40%, preferably a maximum of 10% of the original carbon content remains as organically bound carbon.

Advantageous in the process according to the invention is the continuous or quasi-continuous operation, at least in the secondary oxidation. Quasi-continuously in the sense of the description of the invention means that the primarily oxidized material can also be transported or forced under pressure through the secondary oxidation step.

The conditioning of the primary oxidized materials for the secondary oxidation step is advantageous. It requires a highly homogenous powder matrix in order to finally reach a very uniform retention time during the secondary oxidation. With a uniform and defined retention time for the secondarily oxidized materials as well as the homogenous retention time of the resultant waste gases, a process is achieved which finally enables a pure product free from harmful substances with a justifiable expenditure of effort. An important fluid dynamic parameter in this process is the flow behaviour of the oxidation gases. The flow of the gas defines the powder matrix of the primarily oxidized materials, whereby the gas flow speed lies between 1 mm/s and 4 m/s, preferably between 2 cm/s and 1 m/s.

The conveyor unit with which the primarily oxidized material passes into the secondary temperature treatment zone conditions the material by compressing it, whereby a pressure between 1 kPa and 10.000 kPa is built up. This conditioning/compressing is important for the uniform retention time behaviour and also for the defined flow of the mineral matrix waste gases which are generated during the secondary oxidation. It is additionally advantageous to select the direction of flow of the waste gases so that it is parallel to the direction of movement of the primary oxidate in the secondary oxidation process. Although one could technically strive for a counter-current flow behaviour in the process, there are no practical reasons for doing it. Especially advantageous is a vertical movement of the primary oxidate as well as of the waste gases directed upwards, since in this way the temperature load on the conveyor unit can be restricted.

The conditioning of the primarily oxidized materials could optionally be achieved by sieving off the coarser particles which may have possibly not be completely converted during the secondary oxidation. It is advantageous in this process to choose a sieve that allows only particles smaller than 1 mm, preferably smaller than 0.2 mm to enter into the secondary temperature treatment zone.

As a part of this conditioning, the temperature of the primary oxidate sinks by at least 50° C., preferably by at least 100° C., and particularly preferably by at least 300° C.

During the primary oxidation in accordance with the invention, a volume reduction by at least 65%, preferably by at least 80% takes place, which very much simplifies the handling and the controllability of the process in the following treatment.

The term primary oxidate is understood in the sense of this patent application as the intermediate product which results between the maximum temperature of the primary and the maximum temperature of the secondary oxidation step.

After the second maximum temperature iteration, the product is referred to as a secondary oxidate. In this process, the secondary oxidation is preferably carried out in an externally heated tube or ring aperture through which the primary oxidate is conveyed for example through a rotating auger in a tube. Additionally, a slidegate valve or piston slide system are suitable as a transport system, but also good is a revolving tube, in which the conveyor unit, transport channel and heating zone are united together in one unit, if it is heated simultaneously. It is advantageous if it is heated externally, but directing injecting combustion gases into excess oxygen is also possible.

In addition, it is advantageous to carry out the secondary oxidation using enriched or pure oxygen, water steam or a similar mixture, which lowers the oxidation temperature and the significantly reduces or prevents the formation of NOx or nitrates/nitrites. Here the apparatus is optionally equipped with a gas supply, whereby the suitable oxidation gas/mix is introduced into the oxidation area in a defined fashion. Decisive for the quality of the end product are the retention time behaviour and the flow velocity of the primary oxidate in the secondary temperature treatment zone. The throughput rate for a flying ash oxidation process lies between 0.1 and 4 m/s, and for a fluidized bed process between 0.01 m/h and 3 m/min, preferably between 1 m/h and 1 m/min. Regarding the length of the heating zone for the secondary temperature treatment areas, for a fluidized bed process, this means retention times of 5 s for very small layer thickness of primary oxidate and up to 48 h for very large layer thicknesses. The layer thickness here vary between 1 mm and 50 cm, and are preferably between 5 mm and 5 cm.

For a flying ash oxidation process, typical retention times in the secondary temperature treatment zone lie between 0.1 s and 2 min, preferably between 0.5 and 60 s.

Optionally, if not preferred, the primary oxidation may be carried out in the same apparatus as the secondary oxidation. In accordance with the invention, primary oxidation can also be carried out at a completely different location, for example at the site of the harvest of the basic plant materials. This allows optimization of transport costs to a central secondary oxidation plant. The time interval between primary and secondary oxidation is therefore preferably greater than one day, but if the primary oxidation is continuously carried out, the interval can be only a few seconds, preferably at least 5 s.

It is additionally advantageous to flush the secondary oxidate in water and to treat it with ozone and/or hydrogen peroxide in order to eliminate any traces of organic impurities which may be still present. Optionally in the process of flushing or extracting by a defined addition of CO2 or alkali carbonates or hydrogen carbonates, the formation of difficult to dissolve calcium carbonate is inhibited. This ultimately improves the solubility of the mineral preparation in the body to a considerable level even at high doses and therefore counteracts the hardening of the arteries or the formation of kidney, urine and gall stones.

It is additionally advantageous to make the minerals as bio-available as possible and to separate them from insoluble and absorbable silicic acid, silicon dioxide and silicates. Even when little is still understood about the health aspect of these substances, it can be sensible to separate them from the other minerals.

Additionally is it sensible to set the pH value of the preparations in such a way, that they are bio-compatible. Also the preparation can be broken up into various classes using fractioned extraction: extraction using pure water produces an extract with a high potassium and sodium content. A lightly acidic extraction leads to the enrichment of calcium and magnesium as well as to less easily soluble trace elements such as iron. An alkaline extraction using ammonia leads to a calcium or magnesium-poor preparation, which nevertheless is very rich in trace elements. In this way, different multi-component mineral substance preparations can be specifically produced for various medical indications.

Finally a last production step, which drastically increases the bio-availability of especially the trace elements, is also an option. The following elements are to be considered trace minerals according to their chemical abbreviations:
Li, Rb, Cs, Be, Sr, Ba, Sc, Y, La, Ti, Zr, Hf, V, Nb, Ta, Cr, Mo, W, Mn, Re, Fe, Ru, Os, Co, Rh, Ir, Ni, Pd, Pt, Cu, Ag, Au, Zn, Cd, Ga, In, Ti, Ge, Sn, As, Sb, Bi, Se, Te, Ce, Pr, Sm, Eu, Gd, Tb, Yb, Lu

Trace elements in cluster form are much easier for the body to absorb. For this reason, the diluted alkaline, neutral or acidic solution is optionally treated with at least one reduction agent. Examples of reduction agents include hydrogen, sulphur dioxide, hydrazine, glucose, aldehydes, lactose, etc. The effervescence of hydrogen in the corresponding solution has shown itself to be advantageous. Thereby it is sensible to add a suitable inoculation colloid to the solution, which considerably accelerates the reduction process. These solutions indicate a redox potential of +0.0 V to 2.0 V, preferably of +0.1 V to 1.5 V, especially preferably of 0.2V to 1 V in comparison to a normal hydrogen electrode. The reduction time can be used to control cluster size. The cluster preparations prepared according to the invention consist of 5 to 1,000,000 atoms/ions, preferably of 10 to 8,000 and especially preferably of 12 to 600. The average diameter of the clusters lies between 0.3 nm and 500 nm, preferably between 0.7 and 100 nm. These clusters can consist of one or more trace elements.

Finally the solution can either be concentrated by evaporation and used as a salt or powder in different ways, or the colloidal solution can be used directly, e.g. orally ingested.

The cluster size and also the size of the colloidal particles can be determined via the light refraction on the colloidal solution, amongst other things. Here one may additionally avail oneself of the theories of Rayleigh and of Debye's extended Rayleigh Theory or also of the theory of Zimm, which correlates the relationship between irradiated and diffused light with the particle size of the colloids—for more on this subject, see Physikalische Chemie; W. J. Moore and D. O. Hummel, Walter de Gruyter, 1983, p. 1158 ff. Additional methods for determining cluster size include determining the sedimentation rate in an ultra high speed centrifuge, preferably with optical measuring instrumentation, as well as electrophoresis.

According to the invention, the process can also be carried out in batch mode with two different heating zones, however at least a partially continual process control is preferred, wherein preferably the secondary oxidation is continually running. An application example is illustrated in FIG. 1.

Description of FIG. 1: The primary oxidized material is transported through a rotating auger and pushed through a vertically placed heating zone. This means that the heating zone can be brought up to a temperature of between 300° and 3000° C. using electrical resistances or also inductively or even using electromagnetic radiation or gas or oil firing.

The O2-partial pressure in the primary oxidized material is preferably increased by injecting of oxygen or oxygen-enriched air. This not only reduces the temperature necessary in the heating zone but also considerably reduces the formation of NOx. This finally results in a very slight nitrate/nitrite concentration in the final product in conjunction with the resulting, very uniform and narrow retention time range.

As an alternative to indirect heating, direct heating with gas—preferably with a hydrogen/oxygen mix—can be done, wherein in this example a fly ash oxidation of the primary oxidized material is carried out. However this method is technically more complex because the secondarily oxidized material must again be removed from a much greater volume flow, and furthermore, it requires more energy. Primary oxidation not technically complex. In principle, this process can be carried out in a simple oven or even in a smoke stack.

What is important here is only that the incineration process has already essentially been executed. Only then is it possible to carry out the secondary oxidation in a technically defined manner and to coordinate the simultaneous transport of primary/secondary oxidate and exhaust gases.

A great advantage to this process is also the simple controllability and ratability of the process. Since the waste gas flows completely through the powder matrix of the secondary oxidate, the level of harmful substances in the waste gases corresponds to the level of harmful substances in the product. For this reason, it also is possible to monitor the specific product quite well using an appropriate gas analysis technique, preferably gas chromatography with an attached mass spectroscopic detector with the help of waste gas monitoring.

Furthermore, the process according to the invention encompasses a variant, where the plants are already primarily oxidized at the site of harvest, the primary oxidate is transported to the site of the secondary oxidation and there is further processed according to the process described. The quality of the product can easily be determined by the ratio of the sum of potassium and sodium ions to the nitrate concentration. The latter can be easily analyzed when the nitrate is reduced to nitrite and then sulfanilic acid is added. Due to the intensive coloring of the resulting azo preparation, the nitrate content can easily be determined.

A qualitatively high quality preparation exhibits a (Na+K)/(NO3) ratio (short:NaKNO) greater than 1,000, preferably greater than 10,000, and especially preferably greater than 100,000.

It is not possible to produce NaKNO values greater than 1000 by simply incinerating plant components.

Using the process according to the invention, particularly when pure oxygen flows into the temperature treatment zone, NaKNO values of between 10,000 and 100,000 have been determined.

A further characteristic of the product is the ratio of potassium+sodium to organically-bound carbon: Carbon compounds that survive the high temperatures of production are barely still biologically degradable and are usually extremely toxic. These organic residues are determined using the normal methods used in organic chemistry in the area of trace analysis. Mineral substance preparations produced accordingly can of course be mixed with other minerals, wherein normally at least 2% of the mineral substance preparation produced according to the invention is mixed in with the commercially-available mineral substance preparations, whereby the balance of the commercially-available preparations is clearly increased.

Of course, the quality of any food can also be improved by adding mineral substance preparations produced according to the invention.

Basically all plants and, in this case, also all parts of plants can be used as raw materials; less preferable, but still quite feasible is the use of specific animals or parts of animals such as bone or specific organs. From a medicinal standpoint the use of special plants or parts of animals is therefore important since there are those who would specifically like to implement the “natural” enrichment of a number of trace elements for therapeutic purposes. Especially high quality raw materials include grasses from certain mountain pastures that have not hitherto been intensively cultivated as well as sea algae. Other high quality raw materials include leaves such as olive tree leaves, maple leaves, birch leaves, stinging nettle, aloe vera, thyme, etc. Especially preferable are root and root parts, for example, of wild and/or forest herbs, ginseng, etc. Also fungi and mycelium.

Mineral substances that are produced according to the process described in the invention are suitable as a broad spectrum medication also for remedies for which colloidal silver is recommended. Preparations produced according to the invention are especially effective for the following indications: Heavy metal poisoning, chronic nausea, migraine, allergies, cardio-vascular diseases, high blood pressure. In connection with this, experts have observed a strong correlation between a deficiency in trace elements and the occurrence of disease.

The advantages of the process with respect to prior art can be described in summary as follows:
For the first time multi-component mineral substance preparations of natural origin and free of harmful substances are available. A balanced, natural graduation of minerals and trace elements, as an optimal nutritional supplement to frequently demineralized food, helps refill the body's own mineral substance stores. In this way users strengthen their immune system.

A “natural mineral substance cocktail” is better tolerated during mineralization cures, without health-damaging situations occurring, since no unilateral strain can develop. Due to the high potassium/sodium levels body fluids are alkalized, i.e. the body ph increases and waste products are released resulting in the following: Good blood pressure regulation; prophylactic effect against cardiovascular diseases; Increases the oxygenation of the cells; Increases the energy in the body—increases the level of health and fitness.

### APPLICATION EXAMPLE

Apparatus: An apparatus consisting of one spiral conveyor with a 5 cm long auger, with a diameter of 20 mm, which is equipped with a tube with a diameter of 22 mm. The tube runs horizontally approx. 80 mm and then curves upward, wherein the vertical part amounts to approx. 800 mm. Furthermore in the horizontal area, a gas feed for pure oxygen is attached that is protected from the primary oxidate by a particle filter<0.2 μm. The vertical part is surrounded by a tube furnace; height approx. 500 mm, tube diameter 40 mm. At the end of the vertical tube, a manifold is screwed on that, according to Image 1, carries off the primary oxidate being cooled downwards and draws the exhaust air upwards through an exhaust exit. Furthermore a thermocouple in the heating zone determines the approx. internal temperature in the middle of the tube. A GC with mass spectrometer attached to the exhaust air duct determines the composition of the exhaust air.

Process: 50 kg of dried mountain grasses is burned in a fireplace; the obtained primary oxidate that still smells strongly of burned/smoked material is sieved to 0.2 mm—yield 3.2 kg. The primary oxidate sieved out is placed on the spiral conveyor using a hopper. Furthermore 99.9% pure oxygen at approx. 5 l/min is added to the gas feed. The exterior temperature of the tube in the heating zone has been set to 1200° C. The transport rate in reference to the secondary oxidate amounted to 1-2 g/min. The internal temperature amounted to 825° C. At these parameters, no contaminants were observed in the waste gas, only CO2 and traces of CO. An analysis of the secondary oxidate produced for the presence of nitrate was negative. 10 g of the secondary oxidate produced were dissolved in 1 l of water, decanted, and then filled again with 1 l of hot water and adjusted with citric acid to a pH index of approx. 3. The first extract solution has positive affects on the following: blood pressure; prophylactic effect against cardiovascular diseases; increases the oxygenation of the cells; increases the energy in the body—increases the level of health and fitness.

### APPLICATION EXAMPLE

Apparatus: An apparatus consisting of one spiral conveyor with a 5 cm long auger, with a diameter of 20 mm, which is equipped with a tube with a diameter of 22 mm. The tube runs horizontally approx. 80 mm and then curves upward, wherein the vertical part amounts to approx. 800 mm. Furthermore in the horizontal area, a gas feed for pure oxygen is attached that is protected from the primary oxidate by a particle filter<0.2 μm.

The vertical part is surrounded by a tube furnace; Height approx. 500 mm, tube diameter 40 mm. At the end of the vertical tube, a manifold is screwed on that, according to Image 1, carries off the primary oxidate being cooled downwards and draws the exhaust air upwards through an exhaust exit. Furthermore, a thermocouple in the heating zone determines the approx. internal temperature in the middle of the tube. A GC with mass spectrometer attached to the exhaust air duct determines the composition of the exhaust air.

Process: 50 kg of dried mountain grasses is burned in a fireplace; the obtained primary oxidate that still smelled strongly of burned/smoked material is sieved to 0.2 mm—yield 3.2 kg. The primary oxidate sieved out is placed on the spiral conveyor using a hopper. Furthermore 99.9% pure oxygen at approx. 5 l/min is added to the gas feed. The exterior temperature of the tube in the heating zone has been set to 1200° C. The transport rate in reference to the secondary oxidate amounted to 1-2 g/min. The internal temperature amounted to 825° C. At these parameters, no contaminants were observed in the waste gas, only CO2 and traces of CO. An analysis of the secondary oxidate produced for the presence of nitrate was negative. 10 g of the secondary oxidate produced were dissolved in 1 l of water, decanted, and then filled again with 1 l of hot water and adjusted with citric acid to a pH index of approx. 3. The solution is treated as follows during the extraction:
3: Injection of air, O2-enriched air, pure O2
4: Introduction of primarily oxidized material
5: Drive unit for the spiral conveyor
6: Transport shaft
7: Transport tube
8: Installed components for increasing the blending or improving the heat transfer
9: Secondarily oxidized material
10: Exhaust air tube
11: Waste gas
12: Cycled air
13: Direction of transport

In the following, further aspects of the invention are described:
1. Method for manufacturing multi-component mineral substance preparations free of harmful substances comprising the following steps:
a. Primary oxidation of organic raw materials of plant or animal origin in a primary temperature treatment zone
b. Conditioning of the primarily oxidized material
c. Optional transport of the primarily oxidized material into a secondary temperature treatment zone
d. Secondary oxidation of the primarily oxidized material into a secondary temperature treatment zone
2. Process according to point 1, characterized in that the primarily oxidation is carried out at a temperature of between 500° C. and 3000° C.
3. Process according to at least one of the aforementioned points, characterized in that after the primary oxidation, a maximum 40% of the original carbon content remains as organically bound carbon.
4. Process according to at least one of the aforementioned points, characterized in that gas flows through the matrix of the primary oxidate in the secondary temperature treatment zone.
5. Process according to at least one of the aforementioned points, characterized in that the gas flow rate lies between 1 mm/s and 4 m/s, preferably between 2 cm/s and 1 m/s.
6. Process according to at least one of the aforementioned claims, characterized in that the secondary oxidation is continually carried out.
7. Process according to at least one of the aforementioned points, characterized in that the primarily oxidized material is compressed, wherein a pressure of between 1 kPa and 10,000 kPa is used.
8. Process according to at least one of the aforementioned points, characterized in that the primarily oxidized material, after it has been passed through the primary temperature level, is cooled by at least 50° C.
9. Process according to at least one of the aforementioned points, characterized in that, during primary oxidation, a volume reduction of at least 65% occurs.
10. Process according to at least one of the aforementioned points, characterized in that the primarily oxidized material is graded with a sieve before it is introduced into the secondary temperature level.
11. Process according to at least one of the aforementioned points, characterized in that only particles smaller than 1 mm preferably smaller than 0.2 mm are introduced into the secondary temperature level.
12. Process according to at least one of the aforementioned points, characterized in that a cooling down stage of at least 100° C., preferably more than 300° C. between the primary oxidation and the secondary oxidation occurs.
13. Process according to at least one of the aforementioned points, characterized in that, during primary oxidation, a reduction in organic carbon of about at least 90% and a volume reduction of at least 80% occurs.
14. Process according to at least one of the aforementioned points, characterized in that the primary oxidate in the secondary oxidation level is moved through a channel, for instance, through a tube that is externally heated or that the introduction of energy originates externally.
15. Process according to at least one of the aforementioned points, characterized in that the primary temperature treatment zone and the secondary temperature treatment zone are identical.
16. Process according to at least one of the aforementioned points, characterized in that the primary oxidate as well as the exhaust gases in the second temperature treatment zone are moving in the same direction.
17. Process according to at least one of the aforementioned claims, characterized in that the secondary oxidation is carried out using enriched or pure oxygen and/or water steam.
18. Process according to at least one of the aforementioned points, characterized in that the flow rate of the primary oxidate through the secondary temperature treatment zone amounts to between 4 m/s and 0.01 m/h, based on the area available in the heating zone.
19. Process according to at least one of the aforementioned points, characterized in that, in a fluidized bed process, the flow rate of the primary oxidate through the second temperature treatment zone amounts to preferably between 1 m/min and 1 m/h.
20. Process according to at least one of the aforementioned points, characterized in that the material that has gone through secondary oxidation, in particularly for producing clusters, is partially dissolved or extracted in alkaline, neutral or acidic diluted solutions.
21. Process according to at least one of the aforementioned points, characterized in that ozone and/or hydrogen peroxide is introduced into the diluted solution.
22. Process according to at least one of the aforementioned points, characterized in that the maximum temperatures of the primary oxidation and the secondary oxidation occur longer than a second, preferably longer than 1 minute and especially preferably longer than one day apart.
23. Process according to at least one of the aforementioned points, characterized in that the diluted solution of the secondary oxidation or of the corresponding extract is treated reductively.
24. Process according to point 23, characterized in that the redox potential of the solution shifts about −0.1 to −2 V when measured with a platinum electrode, as opposed to a standard hydrogen electrode.
25. Apparatus for producing mineral substance preparations, comprising:
a. An apparatus for introducing primarily oxidized material of plant or animal origin
b. A conveyor unit for transporting primarily oxidized material
c. A transport channel in which primarily oxidized material is transported
d. A heating zone
e. An exhaust air channel
26. Apparatus according to at least one of the aforementioned points, characterized in that the heating zone is heated to a temperature of between 300° C. and 3000° C.
27. Apparatus according to at least one of the aforementioned points, characterized in that it is continually or quasi-continually operated.
28. Apparatus according to at least one of the aforementioned points, characterized in that it also includes a gas feed.
29. Apparatus according to at least one of the aforementioned points, characterized in that the heating zone is indirectly heated with gas and/or electrically.
30. Apparatus according to at least one of the aforementioned points, characterized in that the primarily oxidized material in the heating zone has a retention time of 5 s-48 h.
31. Mechanism according to at least one of the aforementioned points, characterized in that oxygen gas with an oxygen concentration of at least 90 Vol. % is connected to the gas feed.
32. Mechanism according to at least one of the aforementioned points, characterized in that transport unit, transport channel and heating zone are unified in a heated slew tube.
33. Apparatus according to at least one of the aforementioned points, characterized in that the heating zone has a channel, i.e. tube or annular gap.
34. Apparatus according to at least one of the aforementioned points, characterized in that, in the heating zone, the primarily oxidized material has a coating thickness of between 1 mm and 50 cm, preferably between 5 mm and 5 cm.
35. Apparatus according to at least one of the aforementioned points, characterized in that the primarily oxidized material is heated directly using a gas flame which contains excess oxygen.
36. Apparatus according to at least one of the aforementioned points, characterized in that the primarily oxidized material exhibits a contact time in the heating zone of 0.1 s-5 min., preferably between 0.5 s and 60 s.
37. Apparatus according to at least one of the aforementioned points, characterized in that the conveyer system has a slew tube or spiral conveyor or pushing element.
38. Mineral substance preparations producible or manufactured in accordance with one method according to points 1-24, characterized in that they exhibit a (K+Na)/C(organic) mass ratio of >100, preferably >1,000 and especially preferably >10,000.
39. Mineral substance preparations producible or produced in line with one process according to points 1-24, characterized in that they contain trace elements in colloidal form.
40. Mineral substance preparations according to a least one of the aforementioned points, characterized in that they exhibit a (K+Na)/nitrate ratio of >1000 preferably >10,000 and especially preferably >100,000.
41. Mineral substance preparations according to at least one of the aforementioned points, characterized in that they exhibit a redox potential of +0.0 V to 2.0 V, preferably of +0.1 V to 1.5 V, especially preferably of 0.2 V to 1 V with respect to a standard hydrogen electrode in a diluted solution.
42. Mineral substance preparations according to at least one of the aforementioned points, characterized in that they contain clusters of trace elements.
43. Mineral substance preparations according to at least one of the aforementioned points, characterized in that they consist of one or more trace elements.
44. Mineral substance preparations according to at least one of the aforementioned points, characterized in that the mean diameter of the clusters lies between 0.3 nm and 500 nm, preferably between 0.7 and 100 nm.
45. Mineral substance preparations, according to at least one of the aforementioned points, are characterized in that these clusters comprise of 5 to 1,000,000 atoms/ions, preferably of 10 to 8,000 and especially preferably of 12 to 600.
46. Mineral substance preparations, which according to points 38-45 are characterized in that they are mixed or blended at a maximum of 98% with other minerals and salts or with corresponding mineral substance mixtures in diluted or alcoholic solutions.
47. Mineral substance preparations, according to at least one of the claims 1-24 used as a nutritional supplement, food additive, beauty aid, medication, whereby intake can be in particular either in oral, inhalative, intravenous, rectal or topical form.
48. Mineral substance preparations, according to at least one of the points 38-46 that can be used as a nutritional supplement, food additive, beauty aid, medication, whereby intake can be in particular either in oral, inhalative, intravenous, rectal or topical form.
49. The use of a mineral substance preparation according to either points 47 or 48 for the treatment of heavy metal poisoning, hair loss, chronic nausea, migraine, allergies, cardio-vascular diseases, high blood pressure, wherein mineral substance preparations are prescribed to patients in particular in oral, rectal or intravenous form.

Legend:

The following elements are to be considered trace minerals according to their chemical abbreviations:
Li, Rb, Cs, Be, Sr, Ba, Sc, Y, La, Ti, Zr, Hf, V, Nb, Ta, Cr, Mo, W, Mn, Re, Fe, Ru, Os, Co, Rh, Ir, Ni, Pd, Pt, Cu, Ag, Au, Zn, Cd, Ga, In, Tl, Ge, Sn, As, Sb, Bi, Se, Te, Ce, Pr, Sm, Eu, Gd, Tb, Yb, Lu. 38

1. A process for producing multi-component mineral substance preparations free of harmful substances, the process comprising:
a. performing a primary thermo-oxidative treatment of organic raw materials, of that is one of (i) plant origin and (ii) animal origin, in a primary temperature treatment zone, resulting in primarily oxidized material;
b. conditioning the primarily oxidized material;
c. transporting the product of the primary thermo-oxidative treatment to a second temperature treatment zone; and
d. performing a secondary thermo-oxidative treatment in a second temperature treatment zone.

2-11. (canceled)

12. The process of claim 1,
characterized in that during the conditioning, the coarser particles are sieved off, wherein a sieve is chosen that allows only particles smaller than 1 mm to enter into the secondary temperature treatment zone.

13. The process of claim 1,
characterized in that the primary thermo-oxidative treatment is performed at a temperature of between 500° C. and 3000° C.

14. The process of claim 1,
characterized in that the secondary thermo-oxidative treatment is performed continually.

15. The process of claim 1,
characterized in that the secondary thermo-oxidative treatment is performed quasi-continually.

16. The process of claim 1,
characterized in that the secondary thermo-oxidative treatment is performed using at least one of (i) enriched oxygen and (ii) steam.

17. The process of claim 1,
characterized in that the secondary thermo-oxidative treatment is performed using at least one of (i) pure oxygen and (ii) steam.

18. The process of claim 1,
characterized in that the primarily oxidized material, after it has been passed through the primary temperature level, experiences a cooling by at least 50° C.

19. The process of claim 1,
characterized in that the direction of flow of the waste gases is selected so that it is parallel to the direction of movement of the primary oxidate in the secondary oxidation process.

20. The process of claim 1,
characterized in that the secondary thermo-oxidative treatment is performed at a higher temperature than the primary thermo-oxidative treatment, typically at least 10° C. above the temperature of the primary thermo-oxidative treatment.

21. The process of claim 1 further comprising grinding mechanically the product of the secondary thermo-oxidative treatment.

22. The process of claim 21,
characterized in that the grinding is carried out under dry and/or wet conditions.

23. The process of claim 21 further comprising adding an organic acid to the ground substance in a diluted environment.

24. The process of claim 1,
characterized in that said organic raw materials comprises at least one of vegetables, fruit, grasses, nutshells, lemon peels, orange peels, grapefruit peels, grain brans, the remains of olives, wood, stinging nettle, spinach, and the remains of sugar beets.

25. Mineral substance preparations produced in accordance with a method that comprises:
a. performing a primary thermo-oxidative treatment of organic raw materials, of plant or animal origin, in a primary temperature treatment zone, resulting in primarily oxidized material;
b. conditioning the primarily oxidized material;
c. transporting the product of the primary thermo-oxidative treatment to a second temperature treatment zone; and
d. performing a secondary thermo-oxidative treatment in a second temperature treatment zone;
characterized in that said mineral substance preparations exhibit a (K+Na)/C(organic) mass ratio of greater than 100.

26. The mineral substance preparations of claim 25,
characterized in that said mineral substance preparations exhibit a (K+Na)/nitrate ratio of greater than 1000.

27. The mineral substance preparations of claim 25,
characterized in that said mineral substance preparations contain clusters of trace element, wherein these clusters consist of at least one trace element.

28. The mineral substance preparations of claim 27,
characterized in that the mean diameter of the clusters lies between 0.3 nm and 500 nm.

29. The mineral substance preparations of claim 25, usable as at least one of a nutritional supplement, food additive, beauty aid, and medication, wherein intake can be in one of oral form, inhalative form, intravenous form, rectal form, and topical form.

30. A method for the treatment of heavy metal poisoning, hair loss, chronic nausea, migraine, allergies, cardio-vascular diseases, or high blood pressure, the method comprising prescribing, to a patient, a mineral substance preparation produced in accordance with a method that comprises:
a. performing a primary thermo-oxidative treatment of organic raw materials, of plant or animal origin, in a primary temperature treatment zone, resulting in primarily oxidized material;
b. conditioning the primarily oxidized material;
c. transporting the product of the primary thermo-oxidative treatment to a second temperature treatment zone; and
d. performing a secondary thermo-oxidative treatment in a second temperature treatment zone;
wherein said mineral substance preparation exhibits a (K+Na)/C(organic) mass ratio of greater than 100.
